(19) United States
(12) Patent Application Publication
(10) Pub. No.: US 2019/0263880 A1
(43) Pub. Date: Aug. 29, 2019

(54) PROCESS FOR PREPARATION OF PROTEIN OR PEPTIDE

(71) Applicant: DR. REDDY?S LABORATORIES LIMITED, Hyderabad (IN)

(72) Inventors: Veluri JAGAN MOHAN REDDY, Hyderabad (IN); Mustoori SAIRAM, Hyderabad (IN); Yeruva SRINIVASULU REDDY, Hyderabad (IN); Barui TAPAS, Hyderabad (IN); Jyothishwaran JYOTHISHWARAN, Hyderabad (IN); Kanduri VENKATA SADA SIVA RAO, Hyderabad (IN); Furkhan TAJAMUL, Hyderabad (IN); Malolanarasimhan KRISHNAN, Bangalore (IN); Narayanasamy Murugan RAVICHANDRAN, Hyderabad (IN); Kedari CHAITANYAKUMAR, Hyderabad (IN); Goud Vasker RAKESH, Hyderabad (IN); Desai SAHEJAD, Amreli (IN)

(21) Appl. No.: 16/320,026

(22) PCT Filed: Jul. 25, 2017
(86) PCT No.: PCT/IB2017/054505

§ 371 Date: Jan. 23, 2019

(30) Foreign Application Priority Data
Jul. 27, 2016 (IN) 201641025693

(51) Int. Cl. C07K 14/605; C07K 1/18; C12N 15/62

(52) U.S. Cl. CPC C07K 14/605; C07K 2319/20; C12N 15/62; C07K 1/18

(57) ABSTRACT

The present application relates to a process for the preparation of peptides or proteins or derivatives thereof by expression of synthetic oligonucleotide encoding desired protein or peptide in prokaryotic cell as ubiquitin fusion construct.

### FIELD OF THE INVENTION

The present application relates to a process for the preparation of peptides or proteins or derivatives thereof by expression of synthetic oligonucleotide encoding desired protein or peptide in prokaryotic cell as ubiquitin fusion construct.

### BACKGROUND OF THE INVENTION AND DISCLOSURE OF PRIOR ART

Synthetic peptides are valuable research tools in a variety of biological disciplines. Small peptides are widely used to generate antibodies. Immunologists have found peptides useful for assessing antigenic variation and for studying antigen presentation. Cell biologists employ small peptides to disrupt cell-substrate adhesion and to target proteins to specific cellular compartments. Peptides have long served as model systems in studies on the structure, folding or associations of proteins. Peptides also possess useful therapeutic or pharmacological properties.

Obtaining peptides using solid phase synthetic techniques is a lengthy, laborious and expensive process. In solid phase synthesis, the peptide must be built one residue at a time, with changes of chemicals between each coupling step. Moreover, repetitive couplings become increasingly necessary as the peptide chain lengthens. Purification of the desired synthetic peptide from among truncated or otherwise aberrant synthetic peptides can also prove very troublesome.

The expression of ubiquitin fusion protein or peptide in E. coli and the subsequent purification are described by Monia et al, J. Biol. Chem. 264, 4093-4103 (Mar. 5, 1989). These carboxyl extension proteins (CEP) are from 12 to 80 amino acids in length and are naturally occurring proteins found in various organisms ranging from yeast to humans.

Ecker et al., J. Biol. Chem. 264, 7715-7719 (May 5, 1989) discloses the expression of cloned eukaryotic genes in microorganisms to allow for the isolation of large quantities of naturally occurring protein products which are present in only trace amounts from natural sources.

Butt et al., Proc. Natl. Acad. Sci. 86, 2540-2544 (April 1989) discloses an expression system for cloning ubiquitin-fusion proteins using E. coli and discloses that fusion of ubiquitin by its carboxyl terminal end to the N-terminus of these proteins increases the yield of unstable or poorly expressed proteins such as those referred to by Ecker et al, supra. Butt et al. conclude that ubiquitin fusion technology has the potential for general application in augmenting the yield of cloned gene products in both prokaryotes and eukaryotes.

As early as 1986, Bachmair et al., Science 234, 179-186 (1986), suggests that ubiquitin may be helpful in preparing [beta]-galactosidase fusion proteins having any N-terminal amino acid when expressed in both bacteria and yeast. In the prior art, the emphasis is on the use of both eukaryotic and prokaryotic cell expressions utilizing ubiquitin fusion protein or peptide for the cloning and production of natural intracellular proteins. These natural proteins are often larger than ubiquitin.

Wilkinson et al., Arch. Biochem. Biophys, 250, 390-399 (1986), speculate that ubiquitin may undergo conformational changes following attachment to a target protein.

The objective of the present application is to provide an improved recombinant process for the preparation of peptides or proteins involving the use of ubiquitin fusion tag which addresses the problems associated with the processes reported in prior art discussed above.

### SUMMARY OF THE INVENTION

In the first embodiment, present invention provides a process for producing a protein or peptide or a derivative thereof comprising:
a) expressing the synthetic oligonucleotide encoding desired protein or peptide in host cell as a ubiquitin fusion construct,
b) recovering the expressed ubiquitin fusion protein or peptide.

In first aspect of the first embodiment, the process for producing a protein or peptide or a derivative thereof further comprises:
a) ligating ubiquitin fusion construct, optionally linked to nucleotide encoding affinity tag or SI tag or combination of affinity tag and SI tag in the expression vector;
b) transforming said expression vector having the said ubiquitin fusion construct into host cell and inducing the expression to obtain ubiquitin fusion protein or peptide.

In the second embodiment, the present invention provides a process for producing a protein or peptide or a derivative thereof comprising:
a) expressing the synthetic oligonucleotide encoding desired protein or peptide in host cell as a ubiquitin fusion construct, wherein the said ubiquitin fusion tag is linked to SI tag,
b) recovering the expressed ubiquitin fusion protein or peptide.

In the third embodiment, the present invention provides a synthetic oligonucleotide construct of ubiquitin fusion tag along with an affinity tag and a GLP-1 analogue.

In the fourth embodiment, the present invention provides a synthetic oligonucleotide construct of ubiquitin fusion tag along with GLP-1 analogue and SI tag.

In the fifth embodiment, the present invention provides a synthetic oligonucleotide construct of ubiquitin fusion tag with GLP-1 analogue along with combination of affinity tag and SI tag.

In the sixth embodiment, the present invention provides a synthetic oligonucleotide construct of ubiquitin with Lirapeptide and affinity tag.

In the seventh embodiment, the present invention provides a synthetic oligonucleotide construct of ubiquitin fusion tag along with Lirapeptide and SI tag.

In the eighth embodiment, the present invention provides a synthetic oligonucleotide construct of ubiquitin fusion tag with Lirapeptide along with combination of affinity tag and SI tag.

In the ninth embodiment, the present invention provides a process for expression of fusion protein or peptide in high yield.

In the tenth embodiment, the present invention uses multiple copies of ubiquitin fusion construct cloned together—either under a single promoter or multiple promoters, for the expression. Optionally the multiple copies of ubiquitin fusion construct can be cloned together as a single construct for expression.

In the eleventh embodiment of the present invention, single copy of ubiquitin fusion construct is cloned together for the expression.

In the twelfth embodiment, the present invention provides processes to obtain pure synthetic peptides, by expressing ubiquitin fusion construct in prokaryotic cells, enzymatically cleaving the peptide from ubiquitin and purification of obtained peptide.

In the thirteenth embodiment, the present invention provides a process for producing protein or peptide comprising the steps of:
a) inducing transformant prokaryotic cells which comprise expression vector and ubiquitin fusion construct in fermentation culture medium; wherein the said ubiquitin fusion tag is linked to SI tag,
b) culturing the transformant prokaryotic cells under condition suitable for accumulation of fusion protein;
c) recovering the ubiquitin fusion protein or peptide;
d) optionally, purifying the ubiquitin fusion protein or peptide;
e) enzymatically cleaving the ubiquitin fusion protein or peptide; and
f) recovering the protein or peptide;
g) optionally, purifying the protein or peptide.

In the fourteenth embodiment, the present invention provides a purification of protein or peptide by precipitation at its isoelectric (pI) point.

In the fifteenth embodiment, present invention provides a process for preparing Liraglutide or a derivative thereof comprising the steps of:
a) reacting side chain NH₂ of L-Lysine at the 20^{th} position of Lirapeptide with an acylating agent of formula I,

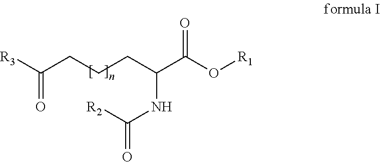

wherein, n is 0-6; R₁ is selected from hydrogen or C₁₋₆ alkyl;
R² is selected from C₃₋₃₉-alkyl, C₃₋₃₉-alkenyl or C₃₋₃₉ alkadienyl;
R³ is selected from hydroxy or a reactive ester thereof such as N-hydroxy imide ester; and
b) optionally, hydrolyzing the acylated Lirapeptide when R¹ is C₁₋₆ alkyl to obtain Liraglutide.

In the sixteenth embodiment, the present invention provides a process for preparing Liraglutide or a derivative thereof comprising the steps of:
a) reacting side chain NH₂ of L-Lysine at the 20^{th} position of Lirapeptide with a compound of formula IV,

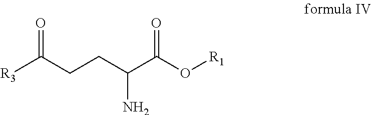

wherein, R1 is selected from hydrogen or C₁₋₆-alkyl; R₃ is selected from hydroxy or a reactive ester thereof such as N-hydroxy imide ester,
b) introducing palmitoyl group on the product obtained in step (a);
c) optionally, hydrolyzing the acylated Lirapeptide obtained in step (b) when R₁ is C₁₋₆ alkyl to obtain Liraglutide.

### BRIEF DESCRIPTION OF THE DRAWINGS

FIG. 1 is Restriction analysis of recombinant constructs. The recombinant plasmids (pUC57-Ubi-Lirapeptide with multiple copies-pentamer) were analyzed by restriction digestion and resolved on 1.2% agarose gel as per loading pattern subsequently stained with EtBr and image captured using UV light.

FIG. 2 is SDS-PAGE analysis of Lirapeptide with ubiquitin fusion construct prepared according to Example 1.

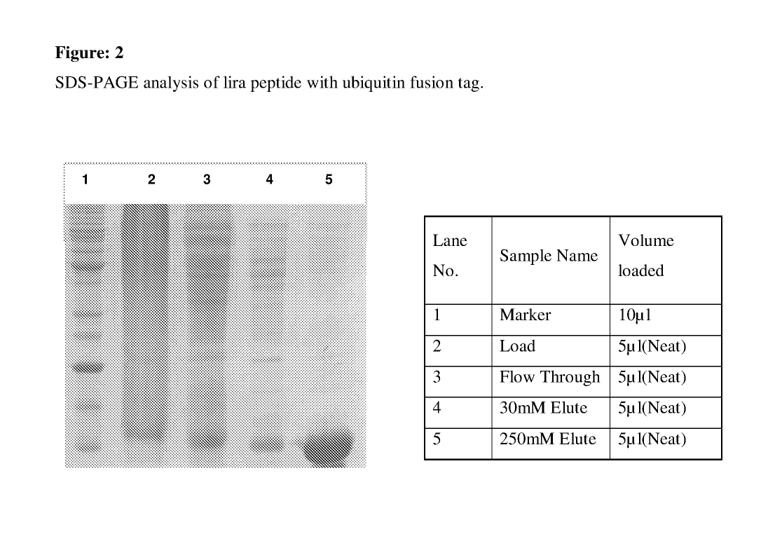

### DETAILED DESCRIPTION

The present application relates to improved recombinant protein production processes through the use of ubiquitin fusion tags for GLP-1 analogues. Ubiquitin is a highly conserved, 76-residue protein having a C-terminus composed of Arg-Gly-Gly and is found in all eukaryotic cells both free and covalently conjugated to a variety of cellular proteins. Ubiquitin is found in cells as diverse as mammals, yeast and celery. Ubiquitin is attached by its carboxyl terminus to amino groups of other proteins. When ubiquitin is attached to the alpha-amino terminus, such products are referred to in the literature as ubiquitin carboxyl extension proteins. In eukaryotic cells, the extension proteins are cleaved from the ubiquitin molecule by hydrolases (peptidases). It has been postulated that attachment of ubiquitin to a protein is a signal for the latter's degradation by proteolysis. Ubiquitin has a neutral isoelectric point of 6.7 and a molecular weight of 8565.

Ubiquitin is extremely stable to heat and extremes of pH which are essential properties for its use as a substrate to facilitate preparation of peptides of the desired amino acid sequence and allow for cleavage by an appropriate enzyme.

For a detailed analysis of ubiquitin, its properties and functions, reference is made to the book “Ubiquitin”, published by Plenum Press, New York (1988) and edited by Martin Rechsteiner.

A variety of peptides having any desired amino acid sequence can be prepared by utilizing ubiquitin as a fusion tag in which synthetic oligonucleotide encoding desired protein or peptide amino-terminal is fused to carboxyl terminal of ubiquitin gene.

The eukaryotic enzymes that cleave peptides from the ubiquitin-peptide fusion products are necessary components in the production of pure peptides. Ubiquitin and the cleavage enzymes are either not present in prokaryotes such as E. coli or are present in such small amounts as not cleavable at the ubiquitin-extension peptide bond. Therefore, cleavage of ubiquitin fusion protein or peptide extended at its carboxyl terminus by synthetic peptides will not occur without the addition of an appropriate cleavage enzyme.

The combination of optimal codons for individual peptide fusions are thought to be in part responsible for the high specific yields which makes this technology so valuable. Focusing on maximizing biomass in the cultures as this invention does, rather than manipulating the physiology of the organism, increases the specific yield, which increases overall yield by decreasing the amount of downstream processing. According to the present invention, amino acid sequence of Lirapeptide has SEQ. ID NO.1 as follows:

1 2 3 4 5 6 7 8 9 10 11 12
His-Ala-Glu-Gly-Thr-Phe-Thr-Ser-Asp-Val-Ser-Ser-
13 14 15 16 17 18 19 20 21 22 23 24
Tyr-Leu-Glu-Gly-Gln-Ala-Ala-Lys-Glu-Phe-Ile-Ala-
25 26 27 28 29 30 31
Trp-Leu-Val-Arg-Gly-Arg-Gly-OH

According to the present invention, amino acid sequence of Liraglutide has SEQ. ID NO.2 as follows:

1 2 3 4 5 6 7 8 9 10 11 12
His-Ala-Glu-Gly-Thr-Phe-Thr-Ser-Asp-Val-Ser-Ser-
13 14 15 16 17 18 19 20 21 22
Tyr-Leu-Glu-Gly-Gln-Ala-Ala-Lys(Pal-Glu)-Glu-Phe-
23 24 25 26 27 28 29 30 31
Ile-Ala-Trp-Leu-Val-Arg-Gly-Arg-Gly-OH

Amino acids may be referred to herein by either their commonly known three letter symbols or by the one-letter symbols recommended by the IUPAC-IUB Biochemical Nomenclature Commission. Likewise nucleotides may be referred to by their commonly accepted single-letter codes.

In the first embodiment, the present invention provides a process for producing a protein or peptide or a derivatives thereof comprising the steps of:
a) expressing the synthetic oligonucleotide encoding desired protein or peptide in host cell as a ubiquitin fusion construct,
b) recovering the expressed ubiquitin fusion protein or peptide.

In an aspect of first embodiment, present invention involves the preparation of ubiquitin fusion construct comprising fusion of amino terminal of synthetic oligonucleotide encoded for desired protein or peptide with carboxyl terminal of ubiquitin. According to present invention, the ubiquitin fusion construct may further fuse with affinity tag or SI tag or combination of affinity tag and SI tag.

In an another aspect of first embodiment, the synthetic oligonucleotide constructs of desired protein or peptide fused with ubiquitin, optionally along with affinity tag or SI tag or combination of affinity tag and SI tag may be cloned in cloning vector. Further, the synthetic construct cloned in cloning vector transformed into cloning prokaryotic cell strains and transformants can be selected by antibiotic selection marker present in cloning vector. The ubiquitin fusion construct isolated by restriction digestion of the recombinant plasmid with the restriction enzymes.

In first aspect of the first embodiment, the process for producing a protein or peptide or a derivative thereof further comprises:
a) ligating ubiquitin fusion construct, optionally linked to nucleotide encoding affinity tag or SI tag or combination of affinity tag and SI tag in expression vector;
b) transforming said expression vector having ubiquitin fusion construct into host cell and inducing the expression to obtain ubiquitin fusion protein or peptide.

According to the step a), the expression vector can be ligated with obtained ubiquitin fusion construct. The expression vector may already have desired restriction sites for ligation or can be introduced into expression vector by using restriction enzyme.

Ligated ubiquitin fusion construct, optionally linked to nucleotide encoding affinity tag or SI tag or combination of affinity tag and SI tag and expression vector can be transformed into host cell for inducing the expression.

In second aspect of the first embodiment, the ligated ubiquitin fusion construct and expression vector optionally can be transformed into cloning prokaryotic strains and transformants can be selected by antibiotic selection marker present in expression vector. The ubiquitin synthetic construct optionally can be purified by gel electrolysis or any other technique well known in the art. The positive colonies of recombinant clone of prokaryotic strains comprising recombinant expression vector (expression vector and ubiquitin fusion construct) initially screened by PCR amplification using primers. After PCR, amplified product of synthetic construct having ubiquitin fusion construct was isolated and finally confirmed by restriction analysis. Optionally, synthetic construct can be purified by gel electrophoresis or any other technique well known in the art.

In third aspect of the first embodiment, the ubiquitin fusion construct is transformed into expression host cell and transformants can be selected by antibiotic selection marker present in expression vector.

According to the step (b) of the first aspect of the first embodiment, protein expression in recombinant expression host is induced by chemical agent. After expression the positive colonies of expression host having fusion protein or peptide can be selected with the help of selection marker and further used for fermentation process.

In the second embodiment, the present invention provides a process for producing a protein or peptide or a derivative thereof comprising:
c) expressing the synthetic oligonucleotide encoding desired protein or peptide in host cell as a ubiquitin fusion construct, wherein the said ubiquitin fusion tag is linked to nucleotide encoding SI tag,
d) recovering the expressed ubiquitin fusion protein or peptide.

In first aspect of second embodiment of the present invention, solubility of ubiquitin fusion construct can be modulated by adding suitable SI tag to N-terminal of ubiquitin fusion protein. The target pI of SI tag fused ubiquitin fusion construct can be from 3.0 to 10.0, preferably from 4-9.

In the second aspect of second embodiment, the present invention provides ubiquitin fusion construct comprising of synthetic oligonucleotide encoding desired protein or peptide; ubiquitin fusion tag and optionally having affinity tag, SI tag, or combination of affinity tag and SI tag.

In the third aspect of second embodiment, the present invention discloses following suitable non-limiting SI tag, which can be fused with ubiquitin fusion construct:

| | Total | | Theoretical PI |
|---|---|---|---|
| | no. of | | value of SI tag |
| | amino | | attached to |
| | acid | | ubiquitin- |
| S. | used | Amino Acid sequence | Lirapeptide |
| No | SI tag | of SI tag | fusion construct |
| 1. | 4 | EDED | 4.99 |
| 2. | 8 | EEDEEDED | 4.65 |
| 3. | 12 | EEDDEDDEEDDE | 4.43 |
| 4. | 16 | EEDDEDDEEDDEEDED | 4.29 |
| 5. | 3 | RRR | 9.05 |
| 6. | 5 | RRRRR | 9.62 |
| 7. | 2 | RR | 8.03 |
| 8. | 3 | AFV | 6.09 |
| 9. | 6 | GAIFLV | 6.09 |
| 10. | 8 | GAIFLGAV | 6.09 |
| 11. | 10 | GAIFLGAVLG | 6.09 |
| 12. | 12 | GAIFLGAVLGVW | 6.09 |
| 13. | 14 | GWAIFLVGAVLGVW | 6.09 |

In fourth aspect of second embodiment, the present invention discloses ubiquitin fusion constructs which can also be designed without affinity tag. The present inventor designed said ubiquitin fusion construct with an aim of purifying fusion protein with non-affinity chromatographic methods. To achieve this, affinity tag of the fusion construct can be replaced with suitable SI tag. SI tag fused ubiquitin construct results in the expression of either partially or completely insoluble fusion protein or either partially or completely soluble fusion protein and this can be purified by inclusion body isolation, solubilization of inclusion body followed by refolding.

The suitable affinity tag can be selected from Polyarginine-tag (Arg-tag), Polyhistidine-tag (His-tag), S-tag, SBP-tag (streptavidin-binding peptide), Maltose binding protein, chitin binding domain (CBD) and the like.

The suitable SI tag is a peptide chain of acidic amino acids, basic amino acid or non-polar amino acid, wherein, chain length is from 2-20 amino acids of acidic amino acid, basic amino acid, non-polar amino acid or suitable combination thereof.

In an aspect, the said SI tag is a peptide chain comprising one or more acidic amino acids selected from Glutamic acid (Glu or E), Aspartic acid (Asp or D), Glycine (Gly or G), or derivatives thereof; wherein chain length is of 1-20 acidic amino acids.

In another aspect, the said SI tag is a peptide chain comprising one or more basic amino acids selected from Lysine (Lys or K), Arginine (Arg or R) or derivatives thereof; wherein chain length is of 1-20 basic amino acids.

In yet another aspect, the said SI tag is a peptide chain comprising one or more non-polar amino acid selected from Glycine (Gly or G), Alanine (Ala or A), Phenylalanine (Pha or F), Valine (Val or V), leucine (Leu or L) or Isoleucine (Ile or I), tryptophan (Trp or W), cysteine (Cys or C) or proline (Pro or P) or derivatives thereof; wherein chain length is of 1-20 non-polar amino acids.

In other aspect of present invention suitable SI tag selected from EDED; EEDEEDED; EEDDEDDEEDDE; EEDDEDDEEDDEEDED; RRR; RRRRR or RR can be used for the expression of fusion protein in soluble form.

In yet other aspect of present invention suitable SI tag selected from AFV; GAIFLV; GAIFLGAV; GAIFLGAVLG; GAIFLGAVLGVW or GWAIFLVGAVLGVW can be used for the expression of fusion protein as insoluble form.

In preferred aspect of present invention SI tag is a peptide chain selected from: acidic amino acids comprising of Glutamine (Glu or E), Aspartic acid (Asp or D) or derivatives thereof; basic amino acids comprising of Lysine (Lys or K), Arginine (Arg or R) or derivatives thereof; non-polar amino acid comprising of Glycine (Gly or G), Alanine (Ala or A), Phenylalanine (Pha or F), Valine (Val or V), leucine (Leu or L), Isoleucine (Ile or I), tryptophan (Trp or W), cysteine (Cys or C), proline (Pro or P) or derivatives thereof; wherein, chain length is from 2-20 amino acids of one or more acidic amino acid, one or more basic amino acid, one or more non-polar amino acid or suitable combination thereof.

The suitable vector used in cloning can be selected from pUC57, pTZ, pBR322 and the like.

The suitable prokaryotic host cell used in cloning can be selected from E. coil, Pseudomonas flurescence, Bacillus subtitis and the like.

The suitable E. coli strains used in cloning can be selected from E. coli DH5a, E. coli Top 10 and the like.

The suitable restriction enzymes used in cloning can be selected from Nde1 and Xho1, Bam HI, SapI, EcoRi, Hind III, Kpnl and the like.

The suitable prokaryotic expression host cells used in expression can be selected from E. coli, Pseudomonas flurescence, Bacillus subtitis and the like.

The suitable E. coli strains used in expression can be selected from E. coli BL21(DE3) pLysS, E. coli JM109, E. coli JM109 (DE3), Rosetta, Origami and the like.

The suitable chemical agent used for induction can be selected from IPTG (isopropylthiogalactoside), tryptophan, nalidexic acid, oxalinic acid, nitrogen or sugars analogs; wherein sugar analogs can be selected from lactose, maltose, arabinose and the like.

The transformation method can be selected from heat shock method, electroporation and the like.

In one variant, heat shock method involves heat shock to cells at about 42° C. for about 30 sec to 120 sec and subsequently kept on ice for about 2-10 minutes.

In an aspect, expression vector that is used in the process of the present invention can be commercially available expression vectors or custom designed vectors selected from pET vectors, pd451sR and the like. pET vectors may be selected from pET24a, pET28a or any other pET vector known to person skill in the art.

In an aspect of the present invention, expression can be carried out at a pH of about 5.0 to about 7.5 and/or at a temperature of about 25° C. to about 42° C.

The expression vector according to present invention comprises of promoters, selection marker, multiple cloning region, origin of replication & operator /repressor system.

The suitable promoter can be selected from T7, TRC, TRP, BAD, LacUV5 or their derivatives or combination thereof.

The suitable selection marker may be selected from kanamycin, ampicillin, chloramphenicol or tetracycline or their combinations in their wild or mutated forms.

The suitable origin of replication can be selected from pUC ORI, pPR322 and the like in their wild type or mutated form.

The suitable Operator/repressor systems can be selected from Lac operon system (see Miller et al. “The operon”, Cold Spring Harbor Laboratory, 1980 and Hillen et al., J. Mol. Biol. 172, 185-201 [1984]).

In the third embodiment, the present invention provides a synthetic oligonucleotide construct of ubiquitin fusion tag along with an affinity tag and a GLP-1 analogue.

In the fourth embodiment, the present invention provides a synthetic oligonucleotide construct of ubiquitin fusion tag along with GLP-1 analogue and SI tag.

In the fifth embodiment, the present invention provides a synthetic oligonucleotide construct of ubiquitin fusion tag with GLP-1 analogue along with combination of affinity tag and SI tag.

In the sixth embodiment, the present invention provides a synthetic oligonucleotide construct of ubiquitin with Lirapeptide and affinity tag.

In the seventh embodiment, the present invention provides a synthetic oligonucleotide construct of ubiquitin fusion tag along with Lirapeptide and SI tag.

In the eighth embodiment, the present invention provides a synthetic oligonucleotide construct of ubiquitin fusion tag with Lirapeptide along with combination of affinity tag and SI tag.

In the ninth embodiment, the present invention provides a process for expression of fusion protein or peptide in high yield.

In the tenth embodiment, the present invention uses multiple copies of ubiquitin fusion construct cloned together for the expression. Optionally the multiple copies of ubiquitin fusion construct can be cloned together as a single construct for expression.

In an aspect of tenth embodiment, the present invention uses two copies of ubiquitin fusion construct cloned together for the expression.

In the eleventh embodiment of the present invention, single copy of ubiquitin fusion construct is cloned together for the expression.

In the twelfth embodiment, the present invention provides processes to obtain pure synthetic peptides, by expressing ubiquitin fusion construct in prokaryotic cells, enzymatically cleaving the peptide from ubiquitin and purification of obtained peptide.

In the thirteenth embodiment, the present invention provides a process for producing protein or peptide comprises:
a) inducing transformant prokaryotic cells which comprise expression vector and ubiquitin fusion construct in fermentation culture medium, wherein the said ubiquitin fusion tag is linked to SI tag;
b) culturing the transformed prokaryotic cells under condition suitable for accumulation of fusion protein;
c) recovering the ubiquitin fusion protein or peptide;
d) optionally, purifying the ubiquitin fusion protein or peptide;
e) enzymatically cleaving the ubiquitin fusion protein or peptide; and
f) recovering the protein or peptide;
g) optionally, purifying the protein or peptide.

In an aspect of thirteenth embodiment, present invention involves fermentation process for increase in accumulation of resulting ubiquitin fusion protein or peptide by inducing transformant prokaryotic cells having expression vector and ubiquitin fusion construct in culture medium with chemical agent.

The fermentation may be carried out in fed-batch or fed-mode to produce ubiquitin fusion protein or peptide. Improved expression of the proteins using the ubiquitin fusion construct of the present invention depends on various parameters of the fermentation process. Some of the suitable parameters are fermentation media, concentration of the inducer, feed media and nutrient feed rate.

The fermentation medium is the medium required for the growth and expression of transformant prokaryotic cells at fermenter scale. Typically the fermentation medium comprises of suitable salts, vitamins, carbon source and nitrogen source. The suitable salts can be selected from ammonium chloride, potassium dihydrogen phosphate, disodium hydrogen phosphate, sodium chloride, calcium chloride, magnesium chloride, EDTA sodium salt, sodium molybdate, zinc sulphate, ferrous sulphate, copper sulphate, monopotassium phosphate, dipotassium phosphate, magnesium sulphate and the like or combination thereof. The carbon source may comprise glucose, glycerol, maltose, sucrose, dextrose, fructose or mannitol and the like or combination thereof. The nitrogen source may comprise ammonia, nitrate, peptone, soya peptone, yeast extract, tryptone and the like or combination thereof. The suitable vitamin can be selected from Thiamine (vitamin B or its related compounds) and the like or combination thereof. The fermentation medium further comprises acids selected from citric acid, boric acid and the like or combination thereof.

The feed medium comprises of salt, carbon source, nitrogen source antibiotics and trace elements. The suitable salt, carbon source and nitrogen source are the same as defined herein above. The feed medium may comprise of antibiotics selected from kanamycin, ampicillin, chloramphenicol, tetracycline and the like and will depend upon the antibiotic marker gene embedded in the vector.

The ubiquitin fusion protein or peptide accumulated in the cytoplasm of the cells can be recovered/harvested by conventional bacterial cell lysis techniques.

An aspect of present invention involves preparation of inclusion body. In one aspect the inclusion body preparation involves re-suspending of cell pellet in non-denaturing lysis buffer comprising of Tris-HCl, phosphate buffer, carbonate buffer and optionally containing chaotropic agents like urea or guanidine hydrochloride.

In another aspect of present invention the inclusion body preparation involves re-suspending of cell pellet in non-denaturing lysis buffer by stirring and treating with lysozyme at room temperature. The re-suspended cells can be homogenized under chilled conditions and centrifuged (Sorvall, Thermofisher). The unbroken cells, large cellular debris, and the inclusion body will be pelleted down and supernatant can be transfer from the pellet. The proteinaceous and non-proteinaceous contaminants present with inclusion body can be removed by washings. The pellet can be re-suspended in wash buffer containing detergents selected from but not limited to sodium deoxycholate, Triton and Tween. The suspension can be centrifuged (Sorvall, Thermofisher). The supernatant containing contaminants can be transfer from the pellet. The inclusion body pellet can be re-suspended in wash buffer comprising of Tris-HCl, NaCl, EDTA and centrifuged. The supernatant containing contaminants can be transferred carefully from the inclusion body pellet. The obtained inclusion body pellet can be solubilized in denaturing buffer selected from Tris-HCl or combination of Tris-HCl with urea or guanidinium chloride.

In an aspect the obtained inclusion body pellet can be solubilized in denaturing buffer (Tris-HCl and urea) and centrifuged. The supernatant can be transfer from the pellet. The obtained supernatant may be optionally clarified by Tangential flow filtration to give ubiquitin fusion protein or peptide.

In an aspect of present invention clarification of lysate by Tangential Flow Filtration (TFF) involves subjecting of above obtained supernatant to TFF using 0.65 μm hollow fiber (Asahi Kasei) system in order to remove insoluble debris and improve clarity.

In another aspect of present invention the cell lysis can be performed without inclusion body preparation. The cell-pellets can be directly re-suspended in denaturing buffer (Tris-HCl, Urea), homogenized and centrifuged. The supernatant can be transfer from the pellet. The obtained supernatant may be optionally clarified by Tangential flow filtration to give clarified supernatant containing ubiquitin fusion protein or peptide.

In an aspect of present invention ubiquitin fusion protein or peptide may be purified by the purification techniques selected from affinity chromatography i.e. Ni NTA chromatography, ion exchange chromatography (cation or anion), reverse phase chromatography or any other technique well known in the art.

In an aspect of present invention ion exchange chromatography can be used for purification of fusion protein or peptide by modulating the isoelectric (pI) point of fusion protein. To modulate the isoelectric (pI) point of fusion protein or peptide between at about 4 and at about 9, the suitable SI tag can be added to the N-terminal of ubiquitin fusion construct.

In the aspect of present invention, cation exchange chromatography can be used for purification when basic amino acid SI tag or non-polar amino acid is used with ubiquitin fusion construct for protein expression.

In the aspect of present invention, anion exchange chromatography can be used for purification when acidic amino acid SI tag or non-polar amino acid is used with ubiquitin fusion construct for protein expression.

In the aspect of present invention, cation exchange chromatography can be used for purification when combination of basic amino acid SI tag or non-polar amino acid and affinity tag with ubiquitin fusion construct is used for protein expression.

In the aspect of present invention, anion exchange chromatography can be used for purification when combination of acidic amino acid SI tag or non-polar amino acid and affinity tag with ubiquitin fusion construct is used for protein expression.

In an aspect of the present invention, cells were harvested and lysed to release the expressed protein in a buffer containing chaotropic agents like urea or guanidine hydrochloride. The purification process generally includes one or two steps to produce a recombinant protein product from crude cell lysates. Another unique feature of preparation of peptides using ubiquitin fusion protein or peptide is that the production is intracellular. When the protein of interest is expressed in insoluble inclusion body, it is easy to separate the inclusion body from soluble materials derived from E. coli such as the proteins of host cell, DNA, polysaccharides in the early stage of purification. According to present invention, the desired peptide is obtained as partly as soluble and partly insoluble form. The protein of interest can be recovered from intracellular proteins by using the difference in charge, solubility, size, hydrophobicity, etc.

In an aspect of the present invention, the ubiquitin fusion protein or peptide can be cleaved enzymatically in vivo or in vitro (using either pure or partially purified fusion specific protease) by cleavage enzyme which cleaves at the junction between ubiquitin fusion tag and the protein or peptide of interest to generate the protein or peptide of interest having the desired amino acid at its amino-terminus. The cleavage enzyme for ubiquitin fusion protein or peptide cleavage can be selected from Yeast ubiquitin hydrolase (YUH1) and the like.

In another aspect of present invention, ubiquitin fusion protein can be directly cleaved enzymatically in vivo or in vitro without any purification, followed by peptide isolation.

In preferred aspect of present invention when insoluble tag is used with ubiquitin fusion construct then purification by anion or cation exchange chromatography of expressed fusion protein can be avoided.

In an aspect of the present invention, expression during fermentation can be carried out at a pH of about 5.0 to about 7.5 and/or at a temperature of about 25° C. to about 42° C.

In ninth embodiment of the present invention, purification of protein or peptide is carried out by precipitation at its isoelectric (pI) point.

In another aspect of ninth embodiment, purification of protein or peptide comprises adjusting the pH of the reaction mixture comprising protein or peptide to its isoelectric (pI) point and isolating the pure protein or peptide.

In an aspect of the present invention, protein or peptide isolated at isoelectric point has a purity of about 80% or about 90% or about 95% as measured by High Performance Liquid Chromatography (HPLC).

In an aspect of the present invention, Lirapeptide isolated at isoelectric point has a purity of about 80% or about 90% or about 95% as measured by High Performance Liquid Chromatography (HPLC).

In a preferred aspect of the present invention, Lirapeptide isolated at isoelectric point has purity of about 80% as measured by High Performance Liquid Chromatography (HPLC).

The desired protein or peptide can be further purified using purification method which can be selected from ion exchange chromatography, affinity chromatography, reversed phase chromatography or any other well-known method in the art.

Ubiquitin fusion systems are employed for the preparation of peptides. They are extremely stable and are expressed at high levels in soluble form or insoluble form. E. coli has been used as a model for ubiquitin-peptide fusion systems in 20-liter batch cultures. Ubiquitin fusion protein or peptide is produced at very high levels in E. coli and related hosts. The specific yield is defined as the percentage, taken as a ratio, of recombinant protein product to total cellular protein, as measured by densitometry of SDS-PAGE gels run on whole cell samples lysed in loading buffer and loaded directly. Specific yields in the E. coli ubiquitin fusion system, grown and induced as described, exceed 20% and approach 30%. The highest reported accumulation of a recombinant protein in E. coli is 50% of the total cellular protein, i.e., 50% specific yield. Another E. coli expression system claims 40% specific yields for some recombinant proteins. Under the conditions specified in the invention, the yield of recovered fusion protein or peptide is one gram to four gram of fusion protein or peptide per liter of bacterial culture. This protein or peptide is both soluble and recoverable. The majority of protein or peptide in supernatants of lysed cells is the product. The fusion can be further purified from host proteins with an 85° C. heat step, in which most of the host proteins precipitate while the ubiquitin fusion stays in solution.

In an aspect of present invention, comprises converting protein or peptide to a derivative thereof.

In another aspect of present invention comprises converting protein or peptide selected from GLP-1 analogue to a derivative thereof.

In preferred aspect of present invention, the said derivative is Liraglutide.

In the fifteenth embodiment, present invention provides a process for preparing Liraglutide or derivatives thereof comprising the steps of:
a) reacting side chain NH₂ of L-Lysine at the 20^{th} position of Lirapeptide with an acylating agent of formula I,

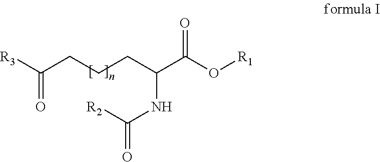

wherein, n is 0-6; R₁ is selected from hydrogen or C₁₋₆ alkyl;
R² is selected from C₃₋₃₉-alkyl, C₃₋₃₉-alkenyl or C₃₋₃₉ alkadienyl;
R³ is selected from hydroxy or a reactive ester thereof such as N-hydroxy imide ester; and
b) optionally, hydrolyzing the acylated Lirapeptide when R₁ is C₁₋₆ alkyl to obtain Liraglutide or derivatives thereof.

In an aspect of fifteenth embodiment, the compound of formula I is selected from a compound of formula II or a compound of formula III:

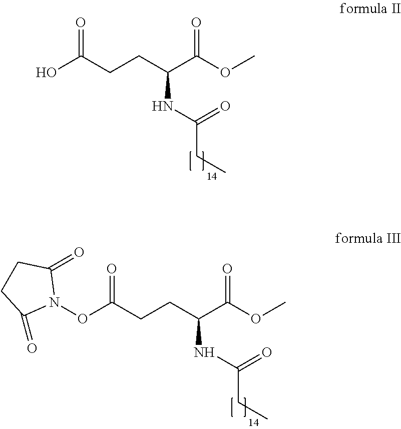

In the sixteenth embodiment, the present invention provides a process for preparing Liraglutide or derivatives thereof comprising the steps of:
a) reacting side chain NH₂ of L-Lysine at the 20^{th} position of Lirapeptide or derivatives thereof with a acylating agent of formula IV,

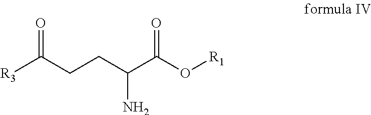

wherein, RI is selected from hydrogen or C₁₋₆-alkyl; R₃ is selected from hydroxy or a reactive ester thereof such as N-hydroxy imide ester,
b) introducing palmitoyl group on the product obtained in step (a);
c) optionally, hydrolyzing the acylated Lirapeptide obtained in step (b) when R₁ is C₁₋₆ alkyl to obtain Liraglutide or derivatives thereof.

In an aspect of the sixteenth embodiment, the compound of formula IV is selected from a compound of formula V or a compound of formula VI:

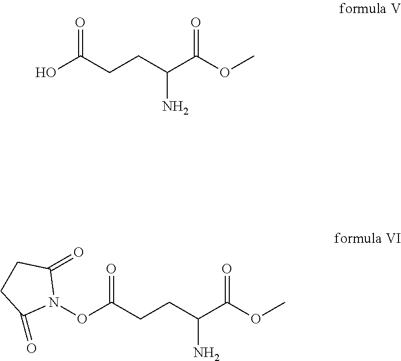 [SMILES: COC(=O)C(N)CCC(=O)O ; COC(=O)C(N)CCC(=O)ON1C(=O)CCC1=O]

In an aspect of the sixteenth embodiment, palmitoyl group can be added to product obtained in step (a) in the presence of coupling reagent and solvent. The palmitoyl group is added by reacting palmitic acid or palmitoyl halide or palmitate ester with glutamate-Lirapeptide that is depicted hereunder,

1 2 3 4 5 6 7 8 9 10 11 12
His-Ala-Glu-Gly-Thr-Phe-Thr-Ser-Asp-Val-Ser-Ser-
13 14 15 16 17 18 19 20 21 22 23
Tyr-Leu-Glu-Gly-Gln-Ala-Ala-Lys(Glu)-Glu-Phe-Ile-
24 25 26 27 28 29 30 31
Ala-Trp-Leu-Val-Arg-Gly-Arg-Gly-OH

The suitable halide for palmitoyl halide can be selected from chloride, bromide or iodide. The palmitate ester may contain alkyl group selected from C₁₋₆ alkyl, e.g. methyl, ethyl, propyl, prop-2-yl, butyl, but-2-yl, 2-methylprop-1-yl, 2-methyl-prop-2-yl (tert-butyl), hexyl and the like.

In an aspect of present invention, acylation of Lirapeptide or derivatives thereof further comprises the steps of:
a) reacting a protein or peptide with a suitable transition metal agent to form a transition metal complex of protein or peptide; and
b) acylating the transition metal complex of protein or peptide with acylating agent selected from compound of formula I or formula II or formula III or formula IV or formula V or formula VI in the presence of coupling reagent and solvent.

In an aspect of present invention, suitable transition metal agent comprising transition metal hydroxide, transition metal carbonate, transition metal chloride, transition metal sulfate, transition metal acetate and the like.

The transition metal can be selected from Scandium (Sc), Titanium(Ti), Vanadium(V), Chromium (Cr), Manganese (Mn), Iron(Fe), Cobalt(Co), Nickel (Ni), Copper (Cu) and the like or combination thereof.

In an aspect of present invention, transition metal agent can be selected from a group comprising copper sulfate, nickel sulfate, nickel acetate, copper acetate and cobalt acetate, cobalt sulfate and the like or combination thereof.

The coupling agent used for the coupling of the amino acids can be selected from HATU, HBTU, EDC, DCC, DIC, BOP and the like or combinations thereof.

In an aspect, additive may be added with coupling reagent which can be selected from HOBt, HOSu, HOAt, and the like or combinations thereof.

The solvent used for the coupling reaction can be selected from dichloromethane, tetrahydrofuran(THF), dimethylformamide (DMF), N-methylpyrolidone, acetonitrile, dimethylsulfoxide (DMSO), and the like or combinations thereof.

In an aspect, the said acylated Lirapeptide obtained in the present invention may contain functional groups such as esters can be selected from C₁₋₆ alkyl, e.g. methyl, ethyl, propyl, prop-2-yl, butyl, but-2-yl, 2-methylprop-1-yl, 2-methyl-prop-2-yl (tert-butyl), hexyl and the like.

According to present invention, ester can be hydrolyzed by basic hydrolysis or acidic hydrolysis. Basic hydrolysis can be carried out using bases such as alkali metal hydroxides including sodium hydroxide, potassium hydroxide, lithium hydroxide and the like; alkali metal carbonates including sodium carbonate, potassium carbonate, sodium bicarbonate, potassium bicarbonate or the like; ammonia, sodium t-butoxide, potassium t-butoxide, sodium methoxide, and the like. The acidic hydrolysis can be carried out using inorganic or organic acids, but are not carboxylic acids. Suitable inorganic acids are those having pKa values below about 4.0 at room temperature in aqueous solution (see Moeller, Inorganic Chemistry, John Wiley & Sons (1952) at pages 314 and 315). Specific examples of such acids are sulfuric acid which is a preferred strong acid catalyst and hydrochloric acid, perchloric acid, nitric acid, phosphoric acid, and hydrofluoric acid. Organic acids suitable for strong acid catalysts herein are noncarboxylic acids having pKa values below 2.0 in water at room temperature (see Handbook of Chemistry and Physics, 58th edition, Chemical Rubber Publishing Company at pages D-150 et seq.). Examples of suitable organic acids are methane sulfonic acid, naphthalene sulfonic acid, trifluoromethyl sulfonic acid, and p-toluene sulfonic acid. Mixtures of strong acid catalysts can also be used.

Suitable solvent that can be used for the said ester hydrolysis includes water; C1-C10 straight or branched chain alcohol such as methanol, ethanol, isopropyl alcohol, 1-butanol, 2-butanol, 2-methyl-2-propanol, 1-pentanol, 2-pentanol, 2,2-dimethyl-1 -propanol, 2,2,2-trimethyl ethanol, 1-decanol, benzyl alcohol; nitriles, such as acetonitrile, propionitrile; ethers such as tetrahydrofuran, dioxane, diisopropylether, diethylether, dibutyl ether, 2-methyltetrahydrofuran, cyclopentyl methyl ether or methyl tert-butyl ether; esters such as ethyl formate, methyl acetate, ethyl acetate, propyl acetate, isopropyl acetate, butyl acetate, methyl propanoate, ethyl propanoate, methyl butanoate or ethyl butanoate; ketones such as acetone, butanone, pentanone, methyl isobutyl ketone; halogenated solvents such as dichloromethane, chloroform, tetrachloromethane, dichloroethane, chlorobenzene or dichlorobenzene; aliphatic hydrocarbon solvents such as methylcyclohexane, cyclohexane, heptane or hexane; aromatic hydrocarbon solvents such as toluene, benzene, chlorobenzene, 4-chlorotoluene, trifluorotoluene, o-xylene, m-xylene or p-xylene; polar aprotic solvents, such as for example, N,N-dimethylformamide, N,N-dimethylacetamide, N-methylpyrrolidone, pyridine, dimethyl sulphoxide, sulpholane, formamide, acetamide, propanamide; or mixtures thereof. The reaction time for ester hydrolysis should be sufficient to complete the reaction which depends on scale and mixing procedures, as is commonly known to one skilled in the art. Typically, the reaction time can vary from about few minutes to several hours. For example the reaction time can be from about 10 minutes to about 24 hours, or any other suitable time period. Suitable temperatures for the said hydrolysis may be less than 120° C., less than 100° C., less than 80° C., less than 60° C., less than 40° C., less than 20° C., less than 10° C., or any other suitable temperatures.

Optionally, the Liraglutide or derivative thereof can be isolated by techniques known in the art. For example, isolation can be done by removal of solvent from the solution containing the product. Suitable techniques which can be used for the removal of solvent include but not limited to evaporation, flash evaporation, simple evaporation, rotational drying, spray drying, agitated thin-film drying, agitated nutsche filter drying, pressure nutsche filter drying, freeze drying or lyophilization or any other technique known in the art. Optionally, in carrying out the processes according to the present invention, the reaction product of a given step can be carried forward to the next step without the isolation of the product from the previous step i.e., one or more reactions in a given process can be carried out in-situ as one pot process optionally in the presence of the same reagent/s used in a previous step wherever appropriate to do so, to make the process of the present invention economical and commercially more viable. The resulting compounds may be optionally further dried. Drying can be carried out in a tray dryer, vacuum oven, air oven, cone vacuum dryer, rotary vacuum dryer, fluidized bed dryer, spin flash dryer, flash dryer, lyophilizer, or the like. The drying can be carried out at temperatures of less than about 60° C., less than about 50° C., less than about 40° C., less than about 30° C., less than about 20° C., or any other suitable temperatures; at atmospheric pressure or under a reduced pressure; as long as the Liraglutide is not degraded in its quality. The drying can be carried out for any desired time until the required product quality is achieved. Suitable time for drying can vary from few minutes to several hours for example from about 30 minutes to about 24 or more hours. Optionally, in carrying out the processes according to the present invention, the reaction product of a given step can be isolated and purified by the methods described herein or the methods known to a person skilled in the art before using in a subsequent step of the process.

An aspect of present invention, purification of protein or peptide can be performed one to five times. The purification processes include but not limited to preparative reverse phase HPLC, ion exchange chromatography, size exclusion chromatography, affinity chromatography or any other well-known technique in the art.

In an aspect of present invention, the purification of protein or peptide comprising the steps of:
a) preparing the sample of protein or peptide in a suitable buffer;
b) optionally, purifying through filter;
c) subjecting the sample through a silica gel column of reverse phase High performance Liquid chromatography (HPLC);
d) eluting the protein or peptide from silica gel column;
e) obtaining, pure protein or peptide.

In an aspect of above step (a), the sample can be prepared by dissolving the crude protein or peptide in suitable buffer.

In an aspect, the suitable buffer that can be used in step (a) can be acidic or basic. The suitable buffer can be selected, but are not limited to Tris (Tris(hydroxymethyl)aminomethane), ammonium acetate, ammonium hydrogen carbonate and the like.

In an aspect of above step (b), the sample of protein or peptide in suitable buffer can be filtered through a filter of about 0.1 μm to about 1 μm.

The Suitable silica gel column types that can be used in above step (c) can be selected from, but are not limited to the following silica gel sorbents: Daisogel™, Kromasil™ C18 100-16, Kromasil™ C18 100-10, Kromasil™ C8 100-16, Kromasil™ C4 100-16, Kromasil™ Phenyl 100-10, Kromasil™ C18 Eternity 100-5, Kromasil™ C4 Eternity 100-5, Chromatorex™ C18 SMB 100-15 HE, Chromatorex™ C8 SMB 100-15 HE, Chromatorex™ C4 SMB 100-15 HE, Daisopak™ SP 120-15 ODS-AP, Daisopak™ SP 120-10-C4-Bio, Daisopak™ SP 200-10-C4-Bio, Zeosphere™ C18 100-15, Zeosphere™ C8 100-15, Zeosphere™ C4 100-15, SepTech ST 150-10 C18, Luna C18 100-10, Gemini C18 110-10, YMC Triart C18 120-5 and YMC Triart C8 200-10.

In an aspect of above step (d), elution of the protein or peptide from silica gel column can be performed by eluent. According to present invention elution can be performed by gradient method or isocratic method. The eluent used in reverse phase High performance Liquid chromatography (HPLC) can be selected from polar solvent, water or suitable mixtures thereof.

In another aspect of above step (d), polar solvent and water can be used at different/independent run time of eluent through the silica gel column of reverse phase High performance Liquid chromatography (HPLC). The polar solvent can be selected from acetonitrile, tetrhydrofuran, acetone, methanol, ethanol, propanol, isopropanol or suitable mixture thereof and the like.

In an aspect of above step (d), modifier can be added to the eluent before elution, wherein modifier is trifluoroacetic acid (TFA).

In another aspect of above step (d), trifluoroacetic acid of about 0.1% to about 0.001% by volume relative to the total volume of the water solution can be used during elution.

In an aspect of above step (d), pure protein or peptide is obtained from pure fractions as collected from reverse phase High performance Liquid chromatography (HPLC) by removing polar solvent and optionally lyophilizing.

In another aspect of above step (d), pure protein or peptide is obtained from pure fractions as collected from reverse phase High performance Liquid chromatography (HPLC) by removing polar solvent from pure fractions and precipitating at isoelectric (pI) point and optionally lyophilizing the obtained pellet.

In other aspect of above step (d), pure protein or peptide is obtained from pure fractions as collected from reverse phase High performance Liquid chromatography (HPLC) by removing polar solvent from pure fractions and lyophilizing.

In an aspect of above step (d), acid can be used for isoelectric (pI) point precipitation. The suitable acid can be selected from an organic acid or an inorganic acid. The suitable organic acid can be selected from formic acid, acetic acid and propionic acid, halogenated acetic acids such as chloroacetic acid, dichloroacetic acid, trifluoroacetic acid and the like or combinations thereof. The suitable inorganic acid can be selected from hydrohalides such as hydrochloric acid, hydrobromic acids, hydrofluoric acid, sulfuric acid, nitric acid or boric acid and the like or combinations thereof.

In another aspect, the isoelectric (pI) point precipitation can be performed one or more times e.g., five times for obtaining pure protein or peptide.

According to the above step (d), pure fraction collected from reverse phase High performance Liquid chromatography (HPLC) can be neutralized by using alkali carbonates selected from sodium carbonate, potassium carbonate, sodium bicarbonate, potassium bicarbonate or the like.

In an aspect of above step (d), protein or peptide purified by reverse phase High Performance Liquid Chromatography (HPLC) has purity at least of about 85%.

In a preferred aspect of the present invention, protein or peptide purified by reverse phase High Performance Liquid Chromatography (HPLC) has purity at least of about 95%.

In another aspect of the present invention, Lirapeptide purified by reverse phase High Performance Liquid Chromatography (HPLC) has purity at least of about 85%.

In a preferred aspect of the present invention, Lirapeptide purified by reverse phase High Performance Liquid Chromatography (HPLC) has purity at least of about 95%.

### Analysis: HPLC

An ACE 5 C18 250×4.6mm, 300A (Part No: ACE-221-2546) was used as a stationary phase. A mobile phase consists of two components; mobile phase A contains 0.1% Trifluoro acetic acid in Mill-Q water and Acetonitrile, mobile phase B contains 0.1% Trifluoro acetic acid in Mill-Q water and Acetonitrile in a gradient mode. The detection was measured at wavelength 220 nm. The injection volume was 10.0 μL. 6M Guanidine HCl is used as a diluent.

### Definitions

The following definitions can be used in connection with the words or phrases used in the present application unless the context indicates otherwise.

The term “amino acid” as used herein refers to an organic compound comprising at least one amino group and at least one acidic group. The amino acid may be a naturally occurring amino acid or be of synthetic origin, or an amino acid derivative or amino acid analog.

The term “amplification” as used herein refers to the production of additional copies of a nucleic acid sequence and is generally carried out using polymerase chain reaction (PCR) technologies well known in the art (Dieffenbach, C. W. and G. S. Dveksler (1995) PCR Primer, a Laboratory Manual, Cold 25 Spring Harbor Press, Plainview, N.Y.).

The term “peptide” as used herein refers to any peptide comprising two or more amino acid residues connected by peptide linkage.

The term “protein” as used here in refers to large molecule composed of one or more chains of amino acids in a specific order.

The term “protein or peptide” as used here in refers to GLP-1 analogues or any other protein or peptide, which contain two or more terminal and/or side chain amino groups.

The term “GLP-1 analogues” as used herein refers to GLP-1 selected from GLP-1(1-35), GLP-1 (1-36), GLP-1(1-36)amide, GLP-1(1-37), GLP-1(1-38), GLP-1(1-39), GLP-1(1-40), GLP-1(1-41) and the like. Preferred GLP-1 include but not limited to Arg²⁶-GLP-1(1-37); Arg³⁴-GLP-1(7-37); Arg³⁴Lys²⁶-GLP-1(7-37); Lys³⁶-GLP-1-(7-37); Arg^{26,34}Lys³⁶-GLP-1(7-37); Arg^{26,34}Lys³⁸GLP-1(7-38); Arg^{26,34}Lys³⁹-GLP-1(7-39); Arg^{26,34}Lys⁴⁰-GLP-1(7-40); Arg²⁶Lys³⁶GLP-1 (7-37); Arg³⁴Lys³⁶-GLP-1 (7-37); Arg²⁶Lys³⁹-GLP-1 (7-39); Arg³⁴Lys⁴⁰GLP-1 (7-40); Arg^{26,34}Lys^{36,39}-GLP-1 (7-39); Arg26,34Lys^{36,40}-GLP-1 (7-40); Gly⁸Arg²⁶-GLP-1 (7-37); Gly⁸Arg³⁴-GLP-1 (7-37); Gly⁸Lys³⁶-GLP-1 (7-37); Gly⁸Arg^{26,34}Lys³⁶GLp-1(7-37); Gly⁸Arg^{26,34}Lys³⁹-GLP-1(7-39); Gly⁸Arg^{26,34}Lys⁴⁰-GLP-1(7-40); Gly⁸Arg²⁶Lys³⁶GLP-1(7-37); Gly⁸Arg³⁴Lys³⁶-GLP-1(7-37); Gly⁸Arg²⁶Lys³⁹-GLP-1(7-39); Gly⁸Arg³⁴Lys⁴⁰GLP-1(7-40); Gly⁸Arg^{26,34}Lys^{36,39}-GLP-1(7-39); Gly⁸Arg^{26,34}Lys^{36,40}-GLP-1(7-40).

The “any other peptide” as mentioned above can be teduglutide.

The term “derivative” is chemically modified protein or peptide or an analogue thereof, wherein at least one substituent is not present in the unmodified protein or peptide or an analogue thereof, i.e. a peptide which has been covalently modified. Typical modifications are amides, carbohydrates, alkyl groups, acyl groups, esters and the like.

The term “acylating” as used herein refers to the introduction of one or more acyl groups covalently bonded to the free amino groups of the protein or peptide.

The term “acylation” means the acylation of the amino group of the protein or peptide.

The term “Lirapeptide” is Arg³⁴-GLP-1(7-37) which is liraglutide before acylation.

The term “Ubi-Lirapeptide” as used herein refers to synthetic oligonucleotide construct of ubiquitin fusion tag and Lirapeptide.

The term “fermentation”, as used herein, is intended to refer to processes involving the production of recombinant protein products.

The term “Pal” is palmitoyl.

The term “HATU” is 2-(7-aza-1H-benzotriazole-1-yl)-1,1,3,3 -tetramethyl uranium hexafluorophosphate.

The term “EDC” is 1-Ethyl-3-(3-dimethylaminopropyl)carbodiimide.

The term “DCC” is Dicyclohexylcarbodiimide.

The term “DIC” is Diisopropylcarbodiimide.

The term “BOP” is Benzotriazol-1-yloxy-tris(dimethylamino)-phosphonium hexafluorophosphate.

The term “HOBt” is 1-Hydroxybenzotriazole.

The term “HOSu” is N-Hydroxysuccinimide.

The term “HOAt” is 1-Hydroxy-7-aza-1H-benzotriazole.

The term “Linker” or “SI Tag” is used synonymously in present specification which means soluble or insoluble tag.

The term “room temperature” as used herein refers to the temperatures of the thing close to or same as that of the space, e.g., the room or fume hood, in which the thing is located'. Typically, room temperature can be from about 20° C. to about 30° C., or about 22° C. to about 27° C., or about 25° C.

The reactions of the processes described herein can be carried out in air or under an inert atmosphere. Typically, reactions containing reagents or products that are substantially reactive with air can be carried out using air sensitive synthetic techniques that are well known to the person skilled in art.

Although the exemplified procedures herein illustrate the practice of the present invention in some of its embodiments, the procedures should not be construed as limiting the scope of the invention. Modifications from consideration of the specification and examples within the ambit of current scientific knowledge will be apparent to one skilled in the art.

### EXAMPLES

### Example 1

### Cloning and Expression of Lirapeptide-Ubiquitin Fusion Construct in E. Coli

Step a. Cloning of Synthetic Gene Construct of Lirapeptide with Ubiquitin Fusion Tag

The synthetic gene construct of Lirapeptide with Ubiquitin fusion tag and 6× His affinity tag was prepared as a synthetic construct and cloned in pUC57. The cloned synthetic construct was transformed by heat shock method into E. coli DH5a and incubated at 37° C. for 1 hr. After incubation, the cells were pellet down and re-suspended. The re-suspended cells were spread over ampicillin medium and incubated overnight (18 hr) to obtain recombinant colonies of E. coli DH5a. The plasmid containing his tag-ubiquitin-Lirapeptide construct was isolated from overnight grown culture of recombinant colonies of E. coli DH5a using Nde1 and Xho1 as restriction digestion enzymes.

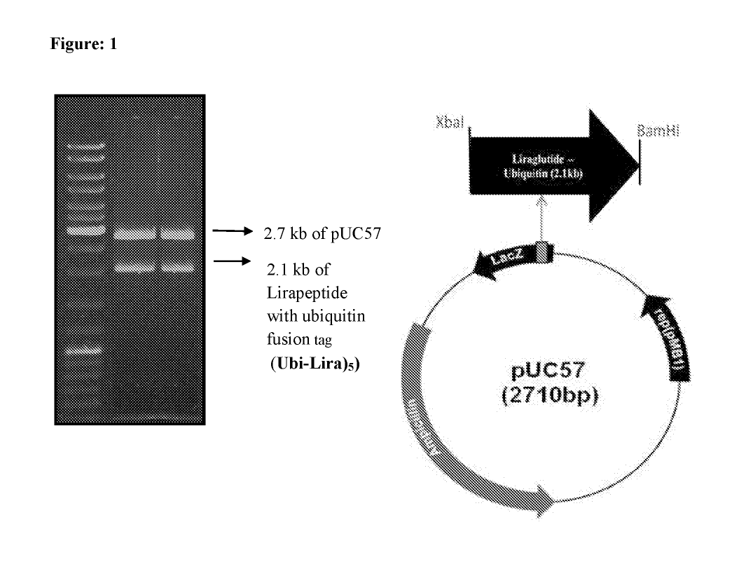

FIG. 1 refers to restriction analysis of recombinant plasmids (pUC57-Ubi-Liraglutide with pentamer copies). The recombinant plasmid was analyzed by restriction digestion (Nde1 and Xho1) and resolved on 1.2% agarose gel as per loading pattern subsequently stained with EtBr and image captured using UV light.

Step b. Sub-cloning & Characterization

The expression vector pET24a was also digested with Nde1 and Xho1 for cohesive end ligation. For ligation, the digested plasmid (pET24a) and the above isolated synthetic construct were ligated with insert: vector in molar ratio of 3:1 was incubated at 16° C. for 18 hr. The ligated product (pET24a: his tag-ubiquitin-Lirapeptide) was transformed in to competent E. coli DH5a by heat shock method and transformants were selected by antibiotic selection marker (Kanamycin (50m/m1)). Positive colonies were initially screened by PCR amplification using T7 primers. After PCR, amplified product of synthetic construct having his tag-ubiquitin-Lirapeptide was resolved on 1.2% agarose gel. Further said synthetic construct was isolated and finally confirmed by restriction enzyme digestion (Nde1-Xho1).

Step c: Expression Studies of Lirapeptide in E. coli Expression Hosts

The recombinant expression plasmids (pET24a +his tag-ubiquitin-Lirapeptide) were transformed into E. coli BL21 (DE3) cells by heat shock method and incubated at 37° C. for 1 hr. After incubation, the cells were pellet down and re-suspended. The re-suspended cells were spread over kanamycin (50 μg/ml) and incubated overnight (18 hr) to obtain isolated recombinant colonies of E. coli BL21 (DE3).

For expression, E. coli BL21 (DE3) were induced with 1 mM IPTG and incubated at 37° C. with shaking overnight (18 hr.). Samples were collected at different intervals (3 hrs, 18 hrs) for checking expression. All induced and un-induced samples were resolved on 1.2% agarose gel. After resolving on gel, the gel was stained with stain (coomassie blue stain) followed by de-staining with de-staining solution (water: methanol: acetic acid). Research cell bank was prepared by inoculating positive colony in 100 ml of culture medium with kanamycin and grown overnight at 37° C. and 250 rpm shaking. Overnight culture was mixed with 50% sterile glycerol and obtain aliquot having recombinant clone of E. coli containing Lirapeptide fusion protein. The obtained aliquot poured into cryovials and stored at −80° C.

### Example 2

### Cloning and Expression of Lirapeptide-Ubiquitin Fusion Construct In E. Coli

Step a. Cloning of Synthetic Gene Construct of Lirapeptide with Ubiquitin Fusion Tag:

The his tag-ubiquitin-Lirapeptide fusion construct was amplified from pUC57-his tag-Ubiquitine-Lirapeptide5 template by PCR using primers, Forward primer (Electra Lira for Sap1-FP-5′-CGC TGA AGC TCT TCT ATG CAC CAT CAC CAT CAT CAC ATG C -3′) Reverse primer (Electra Lira Rav SapI-RP-5′-TTG ACG GCT CTT CTA CCG GAT CCT TAG CCA CGA CCA C -3′). The single copy, two copies and three copies his tag-Ubiquitin-Lirapeptide fusion constructs were amplified and purified using QIAquick gel extraction kit. The gel purified PCR amplicons, single copy, two copies and three copies his tag-ubiquitin-Lirapeptide fusion construct was restriction digested with Sapl and purified using QIA PCR purification kit.

Step b. Sub-Cloning & Characterization

The above example obtained single copy, two copies and three copies his tag-ubiquitin-Lirapeptide fusion construct separately ligated into Sapl site of linear pD451SR expression vector (DNA2.0) and transformed into chemically competent E. coli Top10 by heat shock method and recombinant clones were selected on LB+Kanamycin plates. The transformed clones were confirmed for the presence of Ubiquitin-Lirapeptide fusion protein encoding gene by PCR and restriction digestion.

Step c: Expression Studies of Lirapeptide in E. coli Expression Hosts

The confirmed recombinant plasmids, pD451SR-hisUbiLiral (single copy) pD451SR-hisUbiLira2 (two copies) and pD451SR-hisUbiLira3 (three copies) were transformed into E. coli expression hosts, JM109DE3 and HMS174DE3 by heat shock method and selected on LB+Kanamycin plates. The recombinant E. coli expression clones of JM109 (DE3) and HMS174(DE3) were screened for the expression of Ubiquitin-Lirapeptide fusion protein by inducing with 1 mM IPTG at 37° C. for overnight. Overnight culture was mixed with 50% sterile glycerol and obtain aliquot having recombinant clone of E. coli containing Lirapeptide fusion protein. The obtained aliquot poured into cryovials and stored at −80° C.

### Example 3

### Fermentation Process:

### Step a): Preparation of Pre-Seed:

Pre-seed medium (500 ml) is prepared by dissolving 20 gm/L of yeast extract and 10 gm/L of sodium chloride and dispensed in to a 2 L flask and sterilized. To the sterilized pre-seed medium, 500 μl of kanamycin stock solution (50 mg/ml) was added. 250 μl of glycerol stock having recombinant clone of E. coli containing Lirapeptide fusion construct was inoculated and incubated at 37° C. for 8-12 hours in an incubator shaker.

### Step b): Preparation of Seed Medium In Seed Fermenter:

The following component was used for preparing seed medium in seed fermenter:

TABLE 1

| S. | | | |
|---|---|---|---|
| No | Seed media component | Concentration (g/l) | Quantity |
| 1 | YEAST EXTRACT | 25 | 45 |
| 2 | Sodium Chloride | 10 | 18.01 |
| 3 | Potassium dihydrogen phosphate | 3.3 | 5.95 |
| 4 | Dipotassium phosphate | 7.3 | 13.15 |
| 5 | Magnesium sulphate heptahydrate | 0.2 | 0.366 |
| 6 | Glucose | 6 | 10.88 |

For preparation of seed medium (1.8 L), components (1-4) as per above table no. 1 are weighed and dissolved in DM water(1.5 L) and dispensed in to a 3.0 liter fermenter. Further, components (5-6) mentioned as per table no. 1 were weighed and dissolved in DM water and made up to 200 ml and dispensed in to a 500 ml bottle. 1.8 ml of Kanamycin stock solution was added in the bottle containing Glucose and Magnesium sulphate under aseptic conditions. Further stock solution of bottle having glucose, magnesium sulphate and Kanamycin was transferred in to the seed fermenter. 90 ml of Pre-seed culture medium having recombinant clone of E. coli containing Lirapeptide fusion protein from above step a) was transferred into seed fermenter and seed fermenter was maintained at pH 7, temperature of about 37° C., dissolved oxygen about 25%, and agitation at rate about 300-600 rpm.

### Step c): Preparation of Culture Media for Production Tank Fermenter:

The production tank fermentation medium of 20 liters was prepared according to components mentioned in Table.2 and sterilized. The fermentation medium was poured in production tank fermenter and sterilized.

TABLE 2

| | | Concentration | |
|---|---|---|---|
| S. No | Culture media component | g/L | Quantity (g) |
| 1 | Potassium dihydrogen phosphate | 13 | 260.02 |
| 2 | Diammonium hydrogen phosphate | 4 | 80.01 |
| 3 | Cittric acid anhydrous | 1.7 | 34.01 |
| 4 | Calcium chloride dihydrate | 0.02 | 0.41 |
| 5 | Thiamine hydrochloride | 0.1 | 2.02 |
| 6 | Yeast extract | 3 | 60.03 |
| 7 | EDTA sodium salt | 0.0084 | 0.168 |
| 8 | Cobaltous chloride hexhydrate | 0.0025 | 0.05 |
| 9 | Manganase chloride tetrahydrate | 0.015 | 0.3 |
| 10 | Copper sulphate pentahydrate | 0.0022 | 0.045 |
| 11 | Boric acid | 0.003 | 0.061 |
| 12 | Sodium molybdate dihydrate | 0.0025 | 0.052 |
| 13 | Zinc sulphate heptahydrate | 0.017 | 0.341 |
| 14 | Ferrous sulphate heptahydrate | 0.1 | 2.03 |
| 15 | Magnesium sulphate heptahydrate | 1.2 | 24.02 |
| 16 | Dextrose anhydrous | 10 | 199.98 |
| 17 | Ammonium sulphate | 0.1 | 2.01 |
| 18 | Soyatone | 20 | 400.02 |

### Step c) Preparation of Supplement Component of Fermentation:

1) Glucose (10 g/l; 199.98 gm) and Magnesium sulphate heptahydrate (1.2g/l; 24.02) was prepared in 1 liter RO water.
2) Liquor Ammonium (12.5%) was prepared in RO water of about 1500 ml.
3) Antiform 204 (5%) was prepared in RO water of about 500 ml.

### Step d) Feed Preparation:

Feed medium of 12 liters for fermentation was prepared according to the components of Table 3 and sterilized.

TABLE 3

| | | Quantity | |
|---|---|---|---|
| S.N.O | Feed Component | (g/l) | Quantity (g) |
| 1 | Yeast Extract | 202 | 2424 |
| 2 | Dextrose | 252 | 3024.23 |
| 3 | Magnesium sulphate heptahydrate | 1.51 | 18.123 |
| 4 | Ammonium sulphate | 1.51 | 18.122 |

### Step e) Procedure for Fermentation Process:

After temperature reaches to set point 37° C. of the production tank medium, glucose and magnesium sulphate heptahydrate aseptically was added in bio vessel and adjusted pH 7.0 by using liquor ammonia solution. Further, kanamycin was added to tank media to get final concentration of kanamycin 50 μg/ml and inoculate mature seed (1 L) from seed fermenter into production tank and maintained pH 7.0±0.2 by liquor ammonia; DO₂≥20 -30% and temperature 37±1° C. throughout the batch. Total Batch cycle was used to be 21±3 hrs. Feed was added to fermenter on rise of pH and DO. Further, tank medium was induced by 1M IPTG solution to get final concn 1 mM IPTG, once OD reaches 70±5. Maintain all other parameters same as earlier and maintained for 6-8 hrs after induction. After completion of fermentation, the temperature of broth was decreased to 10-15° C., centrifuged, collected cell mass and stored at −80° C.

### Step f) Lysis of Cell Mass, Solubilization of Fusion Protein and its Clarification By TFF:

100 liter of lysis buffer containing 20 mM tris (242 gm) and 8M Urea (48 kg) was prepared having pH 9.4-9.6 at room temperature. 4000 g of cells were suspended in 30-40 L lysis buffer. Then cells were lysed in a homogenizer and incubated for 2 hours for solubilization. The cell lysate obtained after homogenization was passed through a Hollow fiber tangential flow filtrations (TFF) system and permeate (251) was collected and performed dia-filtration with lysis buffer till permeate wash received around 40-50 L. Permeate and wash permeate was adjusted to 8±0.1 with dilute HCL.

### Example 4

### Purification of Fusion Protein Using Nickel NTA Chromatography:

Permeate (4L) having ubiquitin fusion protein was loaded in to the affinity chromatography (Ni-NTA) which was equilibrated with urea buffer (1M urea+20 mM tris, pH 8.0), at the flow rate of 10 CV/HR. After loading, matrix washed with same urea buffer and started eluting the impurities with elution buffer containing 20 mM Imidazole followed by 200 mM Imidazole to give elute having Ubiquitin fusion protein (8 L). Purity of the fusion protein was 60% as determined RP-HPLC.

### Example 5

### Expression, Lysis & Purification of Lirapeptide-Ubiquitin Fusion Construct In E. coli Using Suitable SI Tag (Soluble Tag)

### Step a) Expression

Ubiquitin fusion construct with suitable SI tag having 4 amino acids (EDED) and 8 amino acids (EEDEEDED) was constructed by PCR amplifying the Ubiquitin fusion gene with specific forward and T7 reverse primer. PCR product was digested with NdeI and XhoI, cloned into appropriately digested pET24a vector. Recombinant plasmid was transformed into E. coli BL21(DE3). Overnight cultures were freshly inoculated into LB with kanamycin and grown in a shaker. Then the expression was induced with 1 mM IPTG at 37° C. Induced cultures along with un-induced controls were analyzed on SDS-PAGE to determine the expression of fusion protein.

### Step b) Cell Lysis:

Expressed cells were suspended in 25 mL of 40 mM Tris-HCl having pH 7.8 of lysis buffer. The lysate was centrifuged at 10,000×g for 30 min and supernatant was collected in a separate tube. The remaining pellet was re-suspended in 25 mL of 40 mM Tris-HCl pH 7.8. Both supernatant and re-suspended pellet were analyzed on SDS-PAGE.

### Step c) Purification:

The above step b) obtained supernatant was subjected to anion exchange chromatography with Q Sepharose Fast Flow. 5 ml matrix was packed into a column and equilibrated with 20 mM Tris-HCl pH 7.7. Then the supernatant was loaded onto the column and flow through was collected. Unbound and loosely bound protein was washed with 20 mM Tris-HCl pH 7.7. Finally fusion protein was eluted with 20 mM Tris-HCl pH 7.7 containing 1M NaCl. Amount of fusion protein obtained from 1 L of culture broth was about 1 g and purity of about 80% as determined by RP-HPLC. The above obtained fusion protein was digested with YUH1 enzyme for the release of peptide from the fusion tag. Digestion was performed at 25° C. and about 50% of the fusion protein was cleaved.

### Example 6

### Expression, Lysis & Purification of Lirapeptide-Ubiquitin Fusion Construct In E. coli Using Suitable SI Tag (Insoluble Tag)

### Step a) Expression

Ubiquitin fusion construct with suitable SI tag having 6 amino acids (GAIFLV) was constructed by PCR amplifying the Ubiquitin fusion gene with specific forward and T7 reverse primer. PCR product was digested with Nde1 and XhoI, cloned into appropriately digested pET24a vector. Recombinant plasmid was transformed into E. coli BL21(DE3). Overnight cultures were freshly inoculated into LB with kanamycin and grown in a shaker. Then the expression was induced with 1 mM IPTG at 37° C.

Induced cultures along with un-induced controls were analyzed on SDS-PAGE to determine the expression of fusion protein.

### Step b) Cell Lysis:

Expressed cells was suspended in 25 mL of 40 mM Tris-HCl having pH 7.8 of lysis buffer. The lysate was centrifuged and supernatant was collected in a separate tube. The remaining pellet was re-suspended in 25 mL of 40 mM Tris-HCl pH 7.8. Both supernatant and re-suspended pellet were analyzed on SDS-PAGE.

### Step c) Purification of Insoluble Fusion Protein:

The above obtained pellet was washed with 0.5% triton X-100 followed by 1% deoxycholtae and finally with 40 mM Tris-HCl pH 7.8. Then the pellet was dissolved in 20 mL of 40 mM Tris-HCl pH 7.8 containing 8M urea. After the pellet was completely dissolved, the solution was centrifuged and supernatant was collected. Supernatant was analyzed on SDS-PAGE for purity and fusion protein quantification. Amount of fusion proteins obtained from 1 L of culture broth was about 1 g and purity of about 80%. The above obtained fusion protein was digested with YUH1 enzyme for the release of peptides from the fusion tag. Digestion was performed at 25° C. and about 50% of the fusion proteins were cleaved.

### Example 7

### Cleavage of Fusion Protein to Obtain Lirapeptide

Ubiquitin hydrolase enzyme (205 ml) was added to 200 mM elute (Ubiquitin fusion protein of 81) from the affinity chromatography in the ratio of 1:20 (20 parts of fusion protein add 1 part of enzyme) for enzymatic digestion and incubated for 4-8 hrs at 30° C. under stirring.

### Example 8

### Purification of Lirapeptide By Isoelectric (pI) Point Precipitation

3.5M Sodium chloride was added to the digestion mixture as obtained in Example 4, and stirred for 30 minutes for dissolution. After dissolution the pH adjusted to 4.7 -4.8 with diluted HCl and sample was incubated for 2-10hrs at 2-8° C. After incubation, sample was centrifuged and collected pellet was washed with acidified water of pH 4.7±0.2 to get lirapeptide pellet (142 gm).

### Example 9

### Lirapeptide Purification By Reverse Phase:

Lirapeptide crude product (10.86 g) was dissolved in 2M urea and 100 mM Tris pH 8.5±0.1 (8.4 L) at a concentration of 1.29 g/L. The solution was filtered through 1.2 μm followed by 0.45 μm and 0.20 μm PP/PES filters. The column packed with Daisogel 40 μm C8 120 A (Dimensions 60×22.8 mm, 644 mL CV) resin was equilibrated with 10% of mobile phase B {Mobile phase B: 100% acetonitrile; Mobile Phase A: 0.3% (v/v) Triethylamine pH 8.5±0.1 (pH adjusted with 5.5 N HCl)};) at a linear flow rate of 127 cm/h before loading. The product was then loaded at a product to resin ratio of 16.86 g/litre of resin or 3.37% g/g of resin. The column was then washed with 2 column volumes (CV) of 10% mobile phase B followed by 3 CV of 28% of mobile phase B at a linear flow rate of 127 cm/h. The Lirapeptide was then eluted with 9 CVs of 28-50% of mobile phase B when fractions (500 mL) were collected. The peak fractions whose purity was greater than 90.00% by analytical HPLC were pooled (4.2 L). Purity of the Elution pool was 94.25% with a recovery of 82%. Typically, acetonitrile present in Elution pool was 30 to 45% (v/v) that was evaporated using rota-vapour at 22° C. and finally carried forward to the next step.

### Example 10

### Preparation of 5-t-butyl-1-methyl palmityl glutamate

In a 2000 mL dry bottom flask, palmitic acid (63.8 g, 248 mmol) was dissolved in 700 mL of dichloromethane at room temperature under argon atmosphere. Triethylamine (42.5 mL, 303 mmol) was then added drop wise and the mixture was stirred for 5 min. 2-(7-aza-1H-benzotriazole-1-yl)-1,1,3,3 -tetramethyl uranium hex afluorophosphate (HATU) (115.59 g, 303 mmol) was added and allowed to stir for additional 10 min. In a separate round bottom flask, L-Glutamic acid tert-butyl ester (70 g, 276 mmol) was taken in 300 mL of dichloromethane and then added dropwise to the above activated palmitic acid in dichloromethane solution. The reaction mixture was allowed to stir at room temperature overnight. The suspension was filtered, quenched with water, extracted with dichloromethane and dried using sodium sulphate. It is then purified by silica gel column chromatography using methanol and dichloromethane to yield 5-t-butyl-1-methyl palmityl glutamate as a white solid (85 g, 180 mmol, 65% yield). ESI-MS (+ve) m/Z: 456.63(MH+).

### Example 11

### Synthesis of 1-methyl palmityl glutamic Acid

Trifluoroacetic acid (139 mL, 1800 mmol) was added to a solution of 5-(t-butyl)-1-methyl palmityl glutamate (82 g, 147 mmol) in dichloromethane (450 mL) and allowed to be stirred at room temperature for 2 h. The resulting solution was quenched with water and extracted using dichloromethane. The solvent was removed under reduced pressure to give 1-methyl palmityl glutamic acid as a dry white solid in quantitative yield. ESI-MS (+ve) m/z: 400.62 (MH+)

### Example 12

### Synthesis of 5-(2, 5-dioxopyrrolidin-1-yl) 1-methyl palmitoyl-L-glutamate

Diisopropylcarbodiimide (DIC, 29 mL, 186 mmol) was added to a solution of 1-methyl palmitoyl glutamic acid (74.5 g, 186 mmol) in tetrahydrofuran (630 mL) at room temperature and stirred for 15 minutes. Further, N-hydroxy succinimide (21.4 g, 186 mmol) was added to the above solution and stirred at room temperature overnight. The suspension was quenched with water (500 mL) extracted with dichloromethane (2000 mL) and dried over anhydrous sodium sulfate. The crude dry solid after evaporation was purified by 40 μm, 750 g of silica Redisep cartridge using Hexane/DCM solvent system at 300 mL/min and obtained 76 g of dry white product (153 mmol, 82%) ESI-MS (+ve) m/z: 497.52 (MH+)

### Example 13

### Conjugation of Lirapeptide with palmityl glutamate Derivatives in the Presence of Copper Sulfate

1.2 M CuSO₄ 5H2O solution (5 μl, 5.9 μmol) was added directly to the lirapeptide (20 mg, 5.9 μmol) and azeotropically dried using toluene and suspended in dimethylformamide (1 mL) at room temperature. Triethylamine was then added for a period of 10 min and the suspension was stirred for 5 min. Tertbutyl dimethyl silyl chloride (4.5 mg, 29.5 μmol) was added to it and stirred for another 10 min. Then, 1-methyl palmityl glutamic acid (2.38 mg, 5.96 μmol) in dimethyl formamide (1 ml) activated with 2-(7-aza-1H-benzotriazole-1-yl)-1,1,3,3 -tetram ethyl uranium hexafluorophosphate (HATU) (2.26 mg, 5.96 μmol) and 2,6-lutidine (1.4 μl, 11.92 μmol) was added drop wise to the above reaction mixture. Additional 6 equivalents of 2, 6-lutidine (4.1 μl, 35.7 μmol) and 2 equivalents of activated 1-methyl palmityl glutamic acid were added to maximize conversion. The progress of the reaction was monitored by UPLC (86% conversion based on lirapeptide area) and the estimated yield by HPLC was 51%.

### Example 14

### Conjugation of Lirapeptide with Palmityl Glutamate Derivatives In the Presence of Nickel Sulfate

NiSO4 6H₂O (3.11 mg, 0.01182 mmol, 1 equiv.) was added directly to the lirapeptide (40 mg, 0.01182 mmol) and azeotropically dried using toluene and suspended in dimethylformamide (2 mL) at room temperature. DMAP (5.8 mg, 0.04728 mmol) was added and the suspension was stirred for 5 min. Then 1-methyl palmityl glutamic acid (4.77 mg, 0.01194 mmol) in dimethyl formamide (0.5 ml) activated with 2-(7-aza-1H-benzotriazole-1-yl)-1,1,3,3-tetramethyl uranium hexafluorophosphate (HATU) (4.54 mg, 0.01194 mmol) and DMAP (2.89 mg, 0.02364 mmol) was added drop wise to the above reaction mixture. An additional equivalent of activated 1-methyl palmityl glutamic acid was added to maximize conversion. The progress of the reaction was monitored by UPLC with 90% conversion in 15 h).

### Example 15

### Conjugation of Lirapeptide with Palmityl Glutamate Derivatives In the Presence of Nickel Acetate Tetrahydrate

Ni(ac)2 4H₂O (2.94 mg, 0.01182 mmol, 1 equiv.) was added directly to the lirapeptide (40 mg, 0.01182 mmol) and azeotropically dried using toluene and suspended in dimethylformamide (2 mL) at room temperature. DMAP (5.8 mg, 0.04728 mmol) was added and the suspension was stirred for 5 min. Then 1-methyl palmityl glutamic acid (4.77 mg, 0.01194 mmol) in dimethyl formamide (0.5 ml) activated with 2-(7-aza-1H-benzotriazole-1-yl)-1,1,3,3-tetramethyl uranium hexafluorophosphate (HATU) (4.54 mg, 0.01194 mmol) and DMAP (2.89 mg, 0.02364 mmol) was added drop wise to the above reaction mixture. An Additional equivalent of activated 1-methyl palmityl glutamic acid was added to maximize conversion. The progress of the reaction was monitored by UPLC with 50% conversion.

### Example 16

### Conjugation of Lirapeptide with Palmityl Glutamate Derivatives In the Presence of Copper Acetate

Cu(ac)2 (2.14 mg, 0.01182 mmol, 1 equiv.) was added directly to the lirapeptide (40 mg, 0.01182 mmol) and azeotropically dried using toluene and dissolved in dimethylformamide (2 mL) at room temperature. DMAP (5.8 mg, 0.04728 mmol) was added and the suspension was stirred for 5 min. Then 1-methyl palmityl glutamic acid (4.77 mg, 0.01194 mmol) in dimethyl formamide (0.5 ml) activated with 2-(7-aza-1H-benzotriazole-1-yl)-1,1,3,3-tetramethyl uranium hexafluorophosphate (HATU) (4.54 mg, 0.01194 mmol) and DMAP (2.89 mg, 0.02364 mmol) were added drop wise to the above reaction mixture. An additional equivalent of activated 1-methyl palmityl glutamic acid was added to maximize conversion. The progress of the reaction was monitored by UPLC with 90% conversion based on lirapeptide area).

### Example 17

### Conjugation of Lirapeptide with Palmityl Glutamate Derivatives In the Presence of Cobalt Acetate

Co(ac)2 (1.05 mg, 0.00591 mmol, 0.5 equiv.) was added directly to the lirapeptide (40 mg, 0.01182 mmol) and azeotropically dried using toluene and dissolved in dimethylformamide (2 mL) at room temperature. DMAP (5.8 mg, 0.04728 mmol) was added and the suspension was stirred for 5 min. Then 1-methyl palmityl glutamic acid (4.77 mg, 0.01194 mmol) in dimethyl formamide (0.5 ml) activated with 2-(7-aza-1H-benzotriazole-1-yl)-1,1,3,3-tetramethyl uranium hexafluorophosphate (HATU) (4.54 mg, 0.01194 mmol) and DMAP (2.89 mg, 0.02364 mmol) was added drop wise to the above reaction mixture. An additional equivalent of activated 1-methyl palmityl glutamic acid was added to maximize conversion. The progress of the reaction was monitored by UPLC with 94% conversion based on lirapeptide area).

### Example 18

### Conjugation of Lirapeptide with N-Hydroxy Succinimide Ester of Palmitoyl Glutamic Acid In an Organic Medium

Triethylamine (0.8 μl, 5.9 μmol) was added to a solution of Lirapeptide (10 mg, 2.95 μmol) in dimethylformamide (1 mL) at room temperature and the suspension was stirred for 5 min. Then, 1.2 M CuSO₄ 5H₂O solution (2.4 ul, 5.9 umol) was added and the mixture was stirred for 10 minutes. Next, tert-Butyldimethyl silyl chloride (2.2 mg, 14.74 μmol) was added to the above reaction mixture and stirred for another 10 min. Finally, 0.08 M solution of N-hexadecanoylglutamic acid γ-N-hydroxysuccinimide ester in N-methyl pyrrolidone (37 μl, 2.95 μmol) and 2,6-Lutidine (2.4 μl, 20.65 μmol) were added and allowed to stirred at room temperature. An additional 2 equivalents of N-hexadecanoylglutamic acid γ-N-hydroxysuccinimide ester (11.8 umol) were used to obtain 72% of the product in the reaction mixture (HPLC data).

### Example 19

### Conjugation of Lirapeptide with N-hydroxy Succinimide Ester of Palmitoyl Glutamic Acid In an Aqueous Medium (process I)

Triethylamine (40 μl, 0.27 mmol) followed by 1.2 M CuSO₄5H₂O solution (493 μl, 0.59 mmol) were added to a solution of lirapeptide (−2 g, 0.59 mmol) in water (400 mL) at 0° C. and stirred for 5 min. Further, additional triethylamine (83 μl, 0.60 mmol) was added to the above reaction mixture in order to maintain the pH ˜9.5. Next, 0.08 M solution of N-hexadecanoylglutamic acid γ-N-hydroxysuccinimide ester in N-methyl pyrrolidone (7.5 mL, 0.59 mmol) was added at 0° C. and slowly warm to room temperature and stirred overnight. Additional 20 μl of Et3N (0.14 mmol) and 440 mg of N-hexadecanoylglutamic acid γ-N-hydroxysuccinimide ester (0.89 μmol) in 11 mL N-methyl pyrrolidone were added to maximize conversion. The progress of the reaction was monitored by UPLC (93% conversion based on lirapeptide area).

### Conjugation of Lirapeptide with N-Hydroxy Succinimide Ester of Palmitoyl Glutamic Acid (process II)

0.5 M CuSO₄5H₂O solution (0.634 g, 2.54 mmol) was added to a solution of lirapeptide (˜34 g, 10.04 mmol) in water (9620 mL) at 12.0 ±1° C. and pH 8, and stirred for 5 min. Further, triethylamine (10.5 mL, 75 mmol) was added to the above reaction mixture in order to maintain the pH ˜10.0. Next, 0.0045 M solution of N-hexadecanoylglutamic acid γ-N-hydroxysuccinimide ester in acetonitrile (4500 mL, 20.08 mmol) was added for 30 minutes using syringe pump at 12.0±1° C. and allowed to stir at that temperature till the reaction showed >95% completion. Additional 10 mL of triethylamine (71.7 mmol) was added during the reaction to maintain the pH ˜10. The HPLC chromatogram showed >95% conversion in 1 h.

### Example 20

### Hydrolysis of Liraglutide Methyl Ester to Obtain Liraglutide (Process I)

Sodium hydroxide solution (5N, 2.5 mL, 12.5 mmol) was added to the crude reaction mixture containing liraglutide methyl ester (2.2 g, 0.59 mmol) in 435 mL of deionized water and the resulting solution was stirred at room temperature, pH 12.8 for 3 h. The completion of reaction was monitored by UPLC and the chromatogram showed the 100% completion of reaction.

### Hydrolysis of Liraglutide Methyl Ester to Obtain Liraglutide (Process II)

237.5 mL of 1.9 M solution of Lithium hydroxide monohydrate (18.9 g, 451 mmol) was added to the crude reaction mixture containing liraglutide methyl ester (10.04 mmol) in 14120 mL of water/acetonitrile solvent mixture (˜31% of MeCN in H₂O₂O) at 8.0±1° C., and the resulting solution was stirred at pH=12.90±0.1 until the LCMS showed >92% conversion.

After the completion of hydrolysis, pH was brought down to ˜8.5 using 52.5 mL of 6N HCl (317 mmol) and 60 mL of 0.25 M EDTA (15 mmol, pH=8.5±0.1) was added and stirred at 8.0 ±1° C. for 1 h. Then pH of the solution was adjusted to 4.7 with 16 mL of 6N HCl and suspension was centrifuged, and the pellet was given for purification.

### Example 21

### Purification of Liraglutide Using High Pressure Reverse Phase Purification System

Liraglutide crude product (14.2 grams of wet pellet) was dissolved in 100 mM Tris pH 8.5±0.1 (913 ml) at a concentration of 1.95g/L. The crude load was filtered through 1.2 μm followed 0.8 μm and 0.45 μm PES filters. The column packed with Dasiogel 10 μm C8 100 Å (Dimensions 50×250mm, 491ml CV) was equilibrated with (10% of mobile phase B (Mobile Phase A: 10 mm Tris pH 8.5±0.1; Mobile phase B: 100% Acetonitrile) at linear flow rate of 230 cm/hr before loading. The peak fractions whose purity was greater than 90.00% by analytical HPLC were pooled (4.2 L). Purity of the Elution pool was 94.25% with a recovery of 82%. Typically, acetonitrile present in Elution pool was 30 to 45% (v/v) that was evaporated using rota-vapour at 22° C. and finally carried forward to the next step. The product was loaded onto the resin at a ratio of 4.36 grams of product/litre of resin or 0.73% gram of product /gram of resin. The column was washed with 3 column volume (CV) of 10% mobile phase B and then with 3 CV of 25% of mobile phases B at a linear flow rate of 230 cm/hr. The Liraglutide was eluted with 15 CVs of 28-42% of mobile phase B at a linear flow rate of 220 cm/hr when fractions (23 ml) were collected. The peak fractions whose purity was greater than 98% by HPLC were pooled (380 ml). Typically, acetonitrile present in elution pool was 31 to 34% (v/v) and was evaporated using rotary evaporator at 22° C., pI precipitated using acetic acid (100μL) and centrifuged at 8500 rpm for 20 min at 5±2° C. Finally, the pellet was lyophilized at 0.16mbar for 24 hours and 1.08 grams of 98.46% pure white powder obtained.

### Example 22

### Purification of Liraglutide Using High Pressure Reverse Phase Purification System

Liraglutide crude product (140 grams of wet pellet) was dissolved in 100 mM Tris pH 8.5±0.1 (4000 ml) at a concentration of 2.34g/L. The crude load was filtered through 1.2 μm followed 0.8 μm and 0.45 μm PES filters. The column packed with Dasiogel 10 μm C8 100 A (Dimensions 100×268mm, 2104 ml CV) was equilibrated with (10% of mobile phase B (Mobile Phase A: 10 mm Tris pH 8.5±0.1; Mobile phase B: 100% Acetonitrile) at a linear flow rate of 220 cm/hr before loading.

The peak fractions whose purity was greater than 90.00% by analytical HPLC were pooled (4.2 L). Purity of the Elution pool was 94.25% with a recovery of 82%. Typically, acetonitrile present in Elution pool was 30 to 45% (v/v) that was evaporated using rota-vapour at 22° C. and finally carried forward to the next step.

The product was then loaded onto the resin at a ratio of 4.45 grams of product/litre of resin or 0.74% gram of product /gram of resin at a linear flow rate of 191 cm/hr. The column was washed with 4 column volumes (CV) of 10% mobile phase B and then with 3 CV of 25% of mobile phases B at a linear flow rate of 220 cm/hr. The Liraglutide was eluted with 18 CVs of 28-40% of mobile phase B at a linear flow rate of 220 cm/hr when fractions (˜220 mL) were collected. The peak fractions whose purity was greater than 98% by HPLC were pooled (2000 mL). Typically, acetonitrile present in Elution pool was 32 to 34% (v/v) and was evaporated using rotary evaporator at 22° C., pI precipitated using HCl (5.5 N, 4.8 mL) and centrifuged at 8000 rpm for 30 min at 5±2° C. Finally, the pellet was lyophilized at 0.04mbar for 24 hours and 5.25 grams of 99.1% pure white powder obtained.

1. A process for producing a protein or peptide or a derivative thereof comprising:
a) expressing the synthetic oligonucleotide encoding desired protein or peptide in host cell as a ubiquitin fusion construct, wherein the said ubiquitin fusion tag is linked to SI tag,
b) recovering the expressed ubiquitin fusion protein or peptide.

2. The process according to claim 1, wherein the SI tag is a peptide chain selected from:
acidic amino acids comprising of Glutamine (Glu or E), Aspartic acid (Asp or D) or derivatives thereof; basic amino acids comprising of Lysine (Lys or K), Arginine (Arg or R) or derivatives thereof; non-polar amino acid comprising of Glycine (Gly or G), Alanine (Ala or A), Phenylalanine (Pha or F), Valine (Val or V), leucine (Leu or L), Isoleucine (Ile or I), tryptophan (Trp or W), cysteine (Cys or C), proline (Pro or P) or derivatives thereof; wherein, chain length is from 2-20 amino acids of one or more acidic amino acid, one or more basic amino acid, one or more non-polar amino acid or suitable combination thereof.

3. The process according to claim 1 wherein suitable SI tag is selected from:
a) EDED
b) EEDEEDED
c) EEDDEDDEEDDE
d) EEDDEDDEEDDEEDED
e) RRR
f) RRRRR
g) RR
h) AFV
i) GAIFLV
j) GAIFLGAV
k) GAIFLGAVLG
l) GAIFLGAVLGVW
m) GWAIFLVGAVLGVW

4. The process according to claim 1, which further comprises increase in accumulation of resulting fusion protein or peptide by fermentation process which comprising:
a) inducing transformant prokaryotic cells which comprise of expression vector having ubiquitin fusion construct in fermentation culture medium; wherein the said ubiquitin fusion tag is linked to SI tag,
b) culturing the transformant prokaryotic cells under condition suitable for accumulation of fusion protein;
c) recovering the ubiquitin fusion protein or peptide;
d) optionally, purifying the ubiquitin fusion protein or peptide;
e) enzymatically cleaving the ubiquitin fusion protein or peptide; and
f) recovering the protein or peptide;
g) optionally, purifying the protein or peptide.

5. The process according to claim 4, wherein the purification of protein or peptide is carried out by anion or cation exchange chromatography.

6. The process according to claim 1, wherein said GLP-1 analogue is Lirapeptide with Seq. ID No. 1.

7. The process according to claim 1, wherein said derivative of peptide is Liraglutide.

8. The process according to claim 7, which comprises:
a) reacting side chain NH₂ of L-Lysine at the 20^{th} position of Lirapeptide with an acylating agent of formula I, 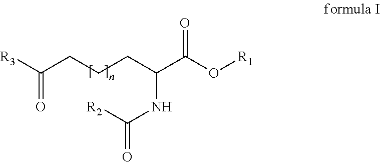
wherein n is 0-6; R₁ is selected from hydrogen or C₁₋₆ alkyl;
R² is selected from C₃₋₃₉-alkyl, C₃₋₃₉-alkenyl or C₃₋₃₉ alkadienyl;
R³ is selected from hydroxy or a reactive ester thereof such as N-hydroxy imide ester; and
b) optionally, hydrolyzing the acylated Lirapeptide when R₁ is C₁₋₆ alkyl to obtain Liraglutide.

9. The process according to claim 8, wherein the compound of formula I is selected from a compound of formula II or a compound of formula III,

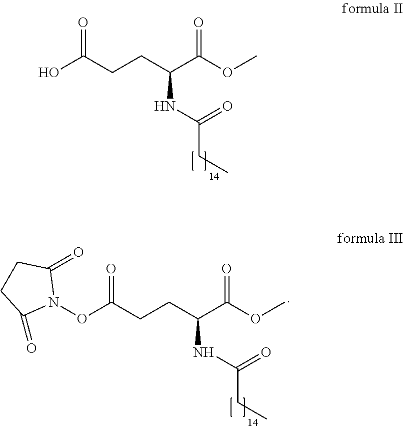

10. The process according to claim 8, which comprises:
a) reacting side chain NH₂ of L-Lysine at the 20^{th} position of Lirapeptide with an acylating agent of formula IV, 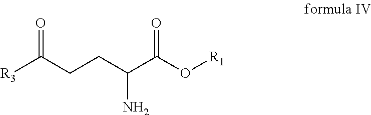
wherein R₁ is selected from hydrogen or C₁₋₁₂-alkyl; R₃ is selected from hydroxy or a reactive ester thereof such as N-hydroxy imide ester,
b) introducing palmitoyl group on the product obtained in step (a);
c) optionally, hydrolysing the acylated Lirapeptide obtained in step (b) when R₁ is C₁₋₆ alkyl to obtain Liraglutide.
